(19) United States
(12) Patent Application Publication
(10) Pub. No.: US 2007/0050011 A1
(43) Pub. Date: Mar. 1, 2007

(54) Lumen-supporting stents and methods for creating lumen-supporting stents with various open/closed designs

(75) Inventors: Richard L. Klein, Santa Rosa, CA (US); James C. Peacock III, San Carlos, CA (US); Michael J. Lee, Santa Rosa, CA (US)

Correspondence Address: PRESTON GATES & ELLIS LLP;ATTN: C. RACHAL WINGER, 925 FOURTH AVE, SUITE 2900, SEATTLE, WA, 98104-1158, US

(73) Assignee: Medlogics Device Corporation, Santa Rosa, CA (US)

(21) Appl. No.: 11/213,385

(22) Filed: Aug. 26, 2005

(51) Int. Cl. A61F 2/06

(52) U.S. Cl. 623/1.16

(57) ABSTRACT

Disclosed herein are various open/closed stent designs and methods for creating the same that can be individually adopted depending on particular treatment objectives. Specifically, the stents of the present invention are manufactured to include different open/closed configurations along their length by varying the number of crossovers, connectors or weld points between sections of the stent. Open portions contain less crossovers, connectors or weld points and are more flexible than closed portions which contain more crossovers, connectors or weld points.

### FIELD OF THE INVENTION

This invention relates to implantable medical devices. More specifically, the invention relates to implantable stents for the treatment or inhibition of stenoses in coronary or peripheral vessels in humans. More specifically, the invention relates to various open/closed stent designs and methods for creating the same.

### BACKGROUND OF THE INVENTION

Cardiovascular disease, including atherosclerosis, is the leading cause of death in the United States. The medical community has developed a number of methods and devices for treating coronary heart disease, some of which are specifically designed to treat the complications resulting from atherosclerosis and other forms of coronary vessel narrowing.

An important development for treating atherosclerosis and other forms of vascular narrowing is percutaneous transluminal angioplasty, hereinafter referred to as “angioplasty.” The objective of angioplasty is to enlarge the lumen of an affected vessel by radial hydraulic expansion. The procedure is accomplished by inflating a balloon within the narrowed lumen of the affected vessel. Radial expansion of the affected vessel occurs in several different dimensions, and is related to the nature of the plaque narrowing the lumen. Soft, fatty plaque deposits are flattened by the balloon, while hardened deposits are cracked and split to enlarge the lumen. The wall of the affected vessel itself is also stretched when the balloon is inflated.

Unfortunately, while the affected vessel can be enlarged thus improving blood flow, in some instances the vessel re-occludes chronically (“restenosis”), or closes down acutely (“abrupt reclosure”), negating the positive effect of the angioplasty procedure. Such restenosis or abrupt reclosure frequently necessitates repeat angioplasty or open heart surgery. While such restenosis or abrupt reclosure does not occur in the majority of cases, it occurs frequently enough that such complications comprise a significant percentage of the overall failures of the angioplasty procedure, for example, twenty-five to thirty-five percent of such failures.

To lessen the risk of restenosis and abrupt closure, various devices have been proposed for mechanically keeping the affected vessel open after completion of the angioplasty procedure. Such endoprostheses (generally referred to as “stents”), are typically inserted into the vessel, positioned across the lesion or stenosis, and then expanded to keep the passageway clear. The stent provides a scaffold which overcomes the natural tendency of the vessel walls of some patients to renarrow, thus maintaining the openness of the vessel and resulting blood flow.

While stents and stent applications of the type described have been found to work well in a number of patients, there is still room for improvement. First, various areas of the vasculature and different treatment sites call for stents with different characteristics. For example, a stent that must travel through a tortuous and highly-curved area of the vasculature to reach a particular treatment site would benefit from enhanced flexibility characteristics that are not necessarily needed in a stent used to treat an easily-accessible treatment site. Likewise, a stent that will be deployed at an area of a vessel that has a branch or bifurcation would benefit from flexibility characteristics not necessarily needed in a stent used to treat a relatively straight and uniform portion of a vessel. Further, as stents are presently used, there can be an abrupt transition between the area of a vessel that is contacted by the stent (and thus receiving the benefits of the stent) and those portions of the vessel that are not. This abrupt transition between stented and unstented portions of a vessel can exacerbate the physiological trauma found at a treatment site. Thus, in some instances, a stent with characteristics that provide for a less abrupt transition between stented and unstented portions of a vessel may be advantageous. Finally, in some instances, it may also be useful to utilize a stent having characteristics that combat restenosis at the treatment site where restenosis is most likely to occur, i.e., the area of the vessel nearest to the proximal (as related to blood flow) end of the stent. Thus, according to the foregoing, there is room for improvement in providing stents with specially designed characteristics that are beneficial at particular treatment sites.

### SUMMARY OF THE INVENTION

The present invention provides methods to create a variety of stent designs that are individually and collectively useful at particular treatment sites requiring stents with particular characteristics. The methods of the present invention provide a variety of stent designs, each useful in different circumstances, by adopting various designs along the length of the stent. Specifically, stents are constructed of repeating “sections.” Adjacent sections of stents are connected to each other by crossovers (in which case the material between sections is continuous), connectors (discrete members connecting adjacent sections) or by weld or fusion points (hereinafter “weld points”). Different designs along the length of a stent are created in accordance with the teachings of the present invention by varying the number of crossovers, connectors or weld points between adjacent sections of a stent. As used herein, open designs have fewer crossovers, connectors, or weld points between adjacent sections and thus create a more flexible area of the stent. Closed designs of the present invention have more crossovers, connectors, or weld points between adjacent sections and thus create a less flexible, more supportive area of the stent. Thus, the number of crossovers, connectors, or weld points is varied to create particular characteristics at different portions of the stent. Importantly, the terms “open” and “closed” are to be interpreted as relative to each other within a particular stent.

One design of the present invention is an “open-closed-open” design. This design provides for greater flexibility at both ends of the stent. This feature can provide for a less abrupt transition between stented and unstented portions of a vessel and can also improve the deliverability of the stent.

A second design of the stents of the present invention includes a “closed-open-closed” design. This stent design can be beneficial when the area to be treated is in the vicinity of (i.e. found before and after) a vessel branch or bifurcation. The open middle portion of the stent provides for greater flexibility so that the stent can conform more readily to the irregular shape of this portion of the vessel. In addition, when treating a vessel in an area of a vessel branch or bifurcation, it is common for the stent to pass over the opening to the second vessel, thereby impeding blood flow into the second vessel (i.e. “gating” the vessel). A more open middle portion can reduce this “gating” effect and allow for better blood flow into the second vessel. Further, if needed, the nature of an open middle portion allows another stent to be deployed through the open middle portion into a gated vessel branch. Closed ends around the open middle portion provide better and more uniform support on each side of the vessel bifurcation.

A third design of the stents of the present invention, the “closed-open” design include stents that are closed at their proximal ends while becoming generally more open along the length of the stent. As used herein, the proximal and distal ends of the stent are to be interpreted as relative to each other and in relation to the distal end of the catheter that delivers the stent to a treatment site. Specifically, the distal end of the stent is closer to the distal end of the delivery catheter than the proximal end is. The “closed-open” design can be advantageous when a more deliverable (i.e. more flexible distal end), yet supported stent is needed at a particular treatment site.

A fourth design of the stents of the present invention includes an “open-closed” design wherein the stents are open at their proximal ends while becoming more closed along the length of the stent. This design can be advantageous when the treatment site is relatively accessible (i.e. a more deliverable stent is not required). The closed distal end of this stent of the present invention provides uniform support while the open proximal end allows for a less abrupt transition between stented and unstented portions of the vessel. An open design at the proximal end of the stent can be especially advantageous because this is the area of a stented vessel most likely to undergo restenosis. A treating physician may choose one of the various embodiments of the stents of the present invention depending on the particular site to be treated and the particular patient's treatment history.

One embodiment of the present invention includes a stent comprising adjacent sections, the sections being connected by crossovers, connectors or weld points wherein the number of crossovers, connectors or weld points connecting the sections is varied between adjacent sections.

Another embodiment of the present invention includes a stent wherein the number of crossovers, connectors or weld points is varied to create an open-closed-open stent.

Another embodiment of the present invention includes an open-closed-open stent having a first open end portion, a closed middle portion and a second open end portion wherein the first open end portion, the closed middle portion and the second open end portion all include the same number of sections and wherein the first open end portion has fewer crossovers, connectors or weld points than the closed middle portion and the second open end portion has fewer crossovers, connectors or weld points than the closed middle portion.

Another embodiment of the present invention includes an open-closed-open stent having three portions: a first open end portion, a closed middle portion and a second open end portion wherein the open end portions each individually have a smaller percentage of crossovers, connectors or weld points than the closed middle portion wherein percentage is the number of crossovers, connectors or weld points within a portion over the possible number of crossovers, connectors or weld points within the portion. In this embodiment, an equal number of sections in each portion is not required.

Another embodiment of the present invention includes a stent wherein the number of crossovers, connectors or weld points is varied to create a closed-open-closed stent.

Another embodiment of the present invention includes a closed-open-closed stent having a first closed end portion, an open middle portion and a second closed end portion wherein the first closed end portion, the open middle portion and the second closed end portion all include the same number of sections and wherein the first closed end portion has more crossovers, connectors or weld points than the open middle portion and the second closed end portion has more crossovers, connectors or weld points than the open middle portion.

Another embodiment of the present invention includes a closed-open-closed stent having three portions: a first closed end portion, an open middle portion and a second closed end portion wherein the closed end portions each individually have a greater percentage of crossovers, connectors or weld points than the open middle portion wherein percentage is the number of crossovers, connectors or weld points within a portion over the possible number of crossovers, connectors or weld points within the portion. In this embodiment, an equal number of sections in each portion is not required.

Another embodiment of the present invention includes a stent wherein the number of crossovers, connectors or weld points is varied to create an open (proximal) to closed (distal) stent.

Another embodiment of the present invention includes an open (proximal) to closed (distal) stent having an open portion and a closed portion wherein the open portion and the closed portion include the same number of sections and wherein the open portion has fewer crossovers, connectors or weld points than the closed portion.

Another embodiment of the present invention includes an open (proximal) to closed (distal) stent having an open portion and a closed portion wherein the open portion has a smaller percentage of crossovers, connectors or weld points than the closed portion wherein percentage is the number of crossovers, connectors or weld points within a portion over the possible number of crossovers, connectors or weld points within the portion. In this embodiment, an equal number of sections in each portion is not required.

Another embodiment of the present invention includes a stent wherein the number of crossovers, connectors or weld points is varied to create a closed (proximal) to open (distal) stent.

Another embodiment of the present invention includes a closed (proximal) to open (distal) stent having an open portion and a closed portion wherein the open portion and the closed portion include the same number of sections and wherein the open portion has fewer crossovers, connectors or weld points than the closed portion.

Another embodiment of the present invention includes a closed (proximal) to open (distal) stent having an open portion and a closed portion wherein the open portion has a smaller percentage of crossovers, connectors or weld points than the closed portion wherein percentage is the number of crossovers, connectors or weld points within a portion over the possible number of crossovers, connectors or weld points within the portion. In this embodiment, an equal number of sections in each portion is not required.

One embodiment of the present invention also includes a method of making a stent with varying characteristics along the length of the stent comprising varying the number of crossovers, connectors or weld points between adjacent sections of the stent.

Another embodiment of the present invention includes a method of making a stent with varying characteristics by varying the number of crossovers, connectors or weld points to create an open-closed-open stent design.

Another embodiment of the present invention includes a method of making a stent with varying characteristics by varying the number of crossovers, connectors or weld points to create a closed-open-closed stent design.

Another embodiment of the present invention includes a method of making a stent with varying characteristics by varying the number of crossovers, connectors or weld points to create an open (proximal) to closed (distal) stent design.

Another embodiment of the present invention includes a method of making a stent with varying characteristics by varying the number of crossovers, connectors or weld points to create a closed (proximal) to open (distal) stent design.

### BRIEF DESCRIPTION OF THE DRAWINGS

FIGS. 1A-1C depict various open-closed-open embodiments of the present invention.

FIGS. 2A and 2B depict various closed-open-closed embodiments of the present invention.

FIGS. 3A and 3B depict various closed (proximal; bottom of FIGS. 3A and 3B) to open (distal; top of FIG. 3A & 3B) embodiments of the present invention.

FIGS. 4A and 4B depict various open (proximal; bottom of FIGS. 4A and 4B) to closed (distal; top of FIG. 4A & 4B) embodiments of the present invention.

FIGS. 5A-5C depict an open-closed-open embodiment (FIG. 5A); an open (proximal; bottom of FIG. 5B) to closed (distal; top of FIG. 5B) embodiment (FIG. 5B) and a closed-open-closed embodiment (FIG. 5C) of the present invention.

FIGS. 6A-6C depict another open-closed-open embodiment (FIG. 6A); another open (proximal; bottom of FIG. 6B) to closed (distal; top of FIG. 6B) embodiment (FIG. 6B) and another closed-open-closed embodiment (FIG. 6C) of the present invention.

### DETAILED DESCRIPTION OF THE INVENTION

U.S. Pat. Nos. 5,292,331 and 5,135,536 to Boneau and Hilstead respectively, and the references cited therein, make it clear that stents can be configured and constructed in many different ways. The present invention is applicable to all known stent designs, and it will be readily apparent from the following discussion of several exemplary designs how the invention can be applied to any type of stent construction.

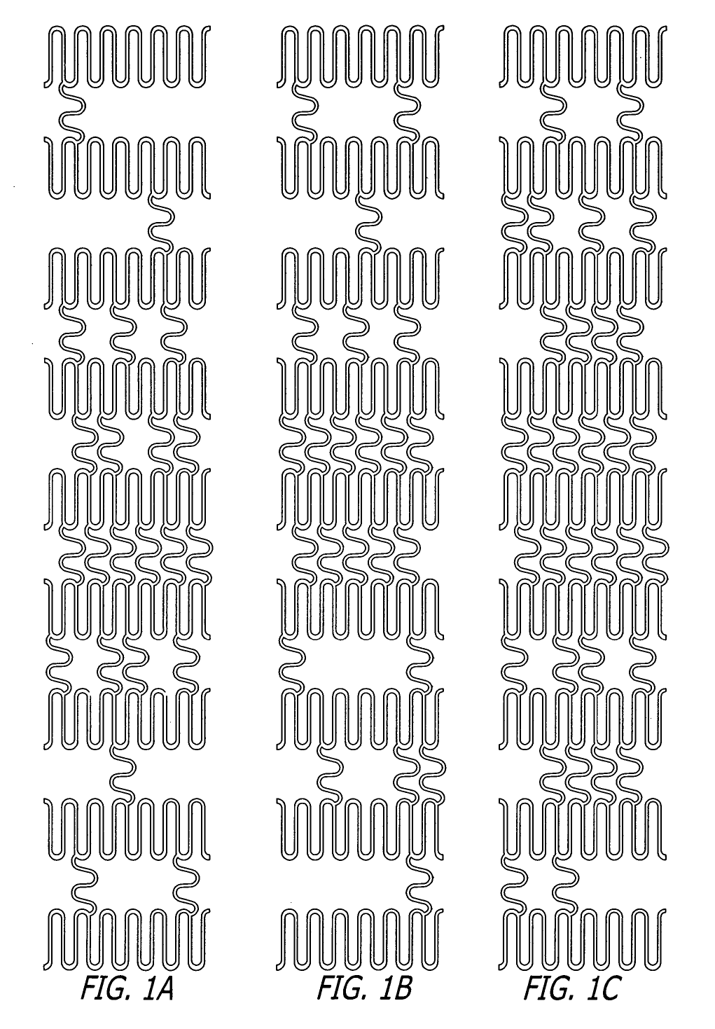

Illustrative stents of the present invention are included in FIGS. 1-6. The sections of the stents of the present invention can have more or less undulations within a section or more or less sections overall than are shown in the FIGS. 1-6, but the simplified depictions shown herein are sufficient to illustrate the present invention. As stated earlier, the terms “open” and “closed” are to be interpreted as relative to each other within a particular stent. Thus, a portion of a stent that is closed in one stent may be “open” when compared to the closed portion of a different stent. This between stent comparison is not appropriate, however, and the closed portion of the stent is defined as such when compared to other portions of the same stent. Further, as will be apparent after a review of the FIGS. 1-6, a transition from open to closed or vice versa need not be uniform as progressing along the length of the stent, but instead can consist of progressions of a more general nature. For example, in a stent comprising a maximum of six crossovers between each section, a progression from open to closed may progress as (in number of connectors between adjacent sections): 1, 1, 2, 2, 3, 3, 4, 4, 5, 5, 6, 6. This progression could also include, however, progressions such as, without limitation, 1, 2, 1, 3, 2, 4, 3, 4, 5, 6, 5, 6 or 2, 1, 3, 1, 2, 4, 3, 5, 6, 4, 6, 5. Finally, the phrases “connector position” or “crossover position” refer to the portions of a stent between sections (for connectors or weld points) or at the intersection of sections (for crossovers) wherein there is an opportunity to modify the number of connectors, crossovers or weld points.

FIGS. 1A, 1B and 1C represent three different embodiments of the present adopting open-closed-open designs. FIGS. 1A-1C depict the following non-numbers of connectors along the length of the stent that create open-closed-open-embodiments:

| Connector | No. of | No. of | No. of |
|---|---|---|---|
| Position | Connectors | Connectors | Connectors |
| 1 | 1 | 2 | 2 |
| 2 | 1 | 1 | 4 |
| 3 | 3 | 3 | 4 |
| 4 | 4 | 6 | 6 |
| 5 | 6 | 5 | 6 |
| 6 | 4 | 2 | 4 |
| 7 | 1 | 3 | 4 |
| 8 | 2 | 1 | 2 |

Thus, as should be apparent from the preceding table, a particular number of crossovers, connectors or weld points at each respective connector position is not required to create a particular embodiment of the present invention. Instead, a variety of different designs can lead to a particular embodiment of the present invention. As a guideline, if a stent design is an open-closed-open design, the stent could be divided into three equal portions (i.e. each portion would contain the same number of crossover, connector or weld point positions). If the number of crossovers, connectors or weld points is counted in each portion, each open end portion should individually have fewer crossovers, connectors or weld points than the middle closed portion. The open end portions need not have the same number of crossovers, connectors or weld points. For instance, while the embodiments depicted in FIGS. 1A-1C cannot be divided into exactly equal thirds for purposes of example, they can divided into open end portion 1 connector positions 1-3), closed middle portion (connector positions 4 and 5) and open end portion 2 (connector positions 6-8). In FIG. 1A, connector positions 1-3 include a total of 5 connectors, connector positions 4 and 5 include a total of 10 connectors and connector positions 6-8 include a total of 7 connectors. In FIG. 1B, connector positions 1-3 include a total of 6 connectors, connector positions 4 and 5 include a total of 11 connectors and connector positions 6-8 include a total of 6 connectors. In FIG. 1C, connector positions 1-3 include a total of 10 connectors, connector positions 4 and 5 include a total of 12 connectors and connector positions 6-8 include a total of 10 connectors. Thus, according to this calculation method, each depicted embodiment is an open-closed-open embodiment of the present invention because each open portion has fewer connectors than the closed portion of its particular stent. Alternatively, to determine if a stent is an open-closed-open design, a stent could be divided into two end portions, whose number of connector positions together combines to create the same number of connector positions of the middle closed portion. With this method, each open end portion should have approximately fewer than half of the number of crossovers, connectors or weld points found within the center closed portion of the stent. This approach can also be adopted in relation to FIGS. 1A-1C. In this approach, connector positions 1-2 and 7-8 constitute open end portions of the stents while connector positions 3-6 constitute closed center portions. In FIG. 1A, connector positions 1 and 2 include a total of 2 connectors and connector positions 7 and 8 include a total of 3 crossovers. These numbers are in comparison to the total of 17 connectors within connector positions 3-6. In FIG. 1B, connector positions 1 and 2 include a total of 3 connectors and connector positions 7 and 8 include a total of 4 crossovers. These numbers are in comparison to the total of 16 connectors within connector positions 3-6. In FIG. 1C, connector positions 1 and 2 and connector positions 7 and 8 both include a total of 6 connectors. These numbers are in comparison to the total of 20 connectors within connector positions 3-6. If the stent were divided so that the number of section numbers in the two end portion areas added together create half of the number of positions of the middle closed portion, each open end portion would have approximately fewer than one-quarter the number of crossovers, connectors or weld points found within the center closed portion of the stent and so forth. Finally, to determine if a particular stent adopts an open-closed-open design, the stent can be divided into two open end portions and a closed middle portion and a percentage of actual crossovers, connectors or weld points over possible spaces for crossovers, connectors or weld points can be calculated. With this calculation method, individual open portions are required to have a smaller percentage of crossovers, connectors or weld points than the particular stent's closed portion.

As stated earlier, stents adopting designs that create an open-closed-open embodiment are useful in providing for a less abrupt transition between stented and unstented portions of a vessel. This is because open portions of the stent provide more flexibility than closed portions of a stent. A less abrupt transition at the proximal end of a stent is especially advantageous because this is an area of a vessel most likely to undergo restenosis after stenting. An open distal end of a stent is advantageous in addition to providing for a less abrupt transition between stented and unstented portions of a vessel because it can improve the deliverability of a stent. Improved deliverability is especially advantageous when a stent must navigate tortuous or highly curved vessels to reach a particular treatment site.

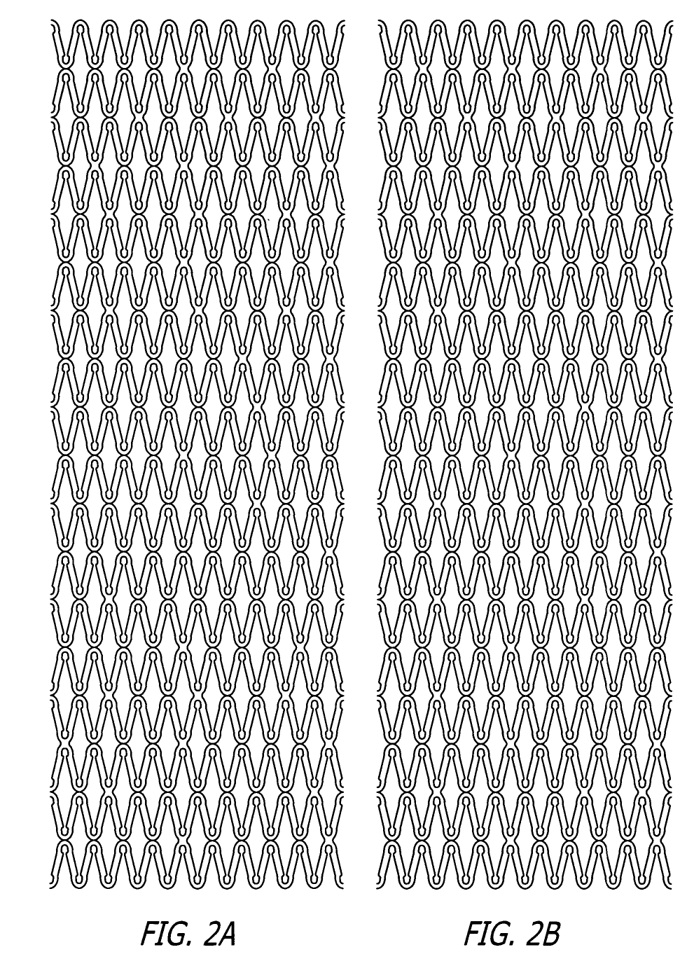

FIGS. 2A and 2B represent non-limiting closed-open-closed embodiments of the present invention. FIGS. 2A and 2B represent embodiments of the present invention employing crossovers between adjacent sections of the stent. Thus, these FIGS. 2A and 2B are different than the stent configurations depicted in FIGS. 1A-1C, illustrating that the present invention can be applied to different stent configurations. FIGS. 2A-2B depict the following non-limiting numbers of crossovers at each crossover position along the length of the stent that create closed-open-closed embodiments:

| Crossover | No. of | No. of |
|---|---|---|
| Position | Crossovers | Crossovers |
| 1 | 2 | 3 |
| 2 | 3 | 3 |
| 3 | 3 | 2 |
| 4 | 3 | 2 |
| 5 | 2 | 2 |
| 6 | 3 | 1 |
| 7 | 2 | 1 |
| 8 | 1 | 1 |
| 9 | 2 | 1 |
| 10 | 1 | 1 |
| 11 | 1 | 1 |
| 12 | 1 | 1 |
| 13 | 2 | 1 |
| 14 | 3 | 1 |
| 15 | 3 | 2 |
| 16 | 2 | 2 |
| 17 | 2 | 2 |

As a guideline to determine whether a particular stent adopts a closed-open-closed design, the stent could be divided into three equal portions (i.e. same number of crossover positions). If the number of crossovers, connectors or weld points is counted in each portion, each closed end portion should individually have at least one more crossover, connector or weld point than the middle open portion. The closed end portions need not have the same number of crossovers, connectors or weld points. Alternatively, to determine if a stent is a closed-open-closed design, a stent could be divided into end portions, whose number of crossover positions together is the same or different from the number of crossover positions of the middle open portion. With this method, regardless of its size, each closed end portion should have a greater percentage of crossovers/possible crossovers, connectors or weld points compared to the percentage of crossovers/possible crossovers, connectors or weld points found within the open middle portion. Percentage refers to the actual number of crossovers, connectors or weld points compared to the spaces for possible crossovers, connectors or weld points.

As stated earlier, stents adopting a closed-open-closed design are useful for treatment that include a vessel branch or bifurcation. The open middle portion provides for greater flexibility at the irregular shape of the branch or bifurcation. If the open middle portion gates a side branch or one side of a vessel bifurcation, the open configuration can be opened further through balloon inflation to promote blood flow to the gated vessel. In addition to having an open middle portion, in one embodiment, this stent design can also include longer stent sections in the middle of the stent which could increase the flexability of this portion of the stent further. Finally, a second stent could be deployed through the open middle portion into the second vessel stemming from the branch or burification. The closed ends of this stent design provide additional support for the vessel on both sides of the flexible and open center.

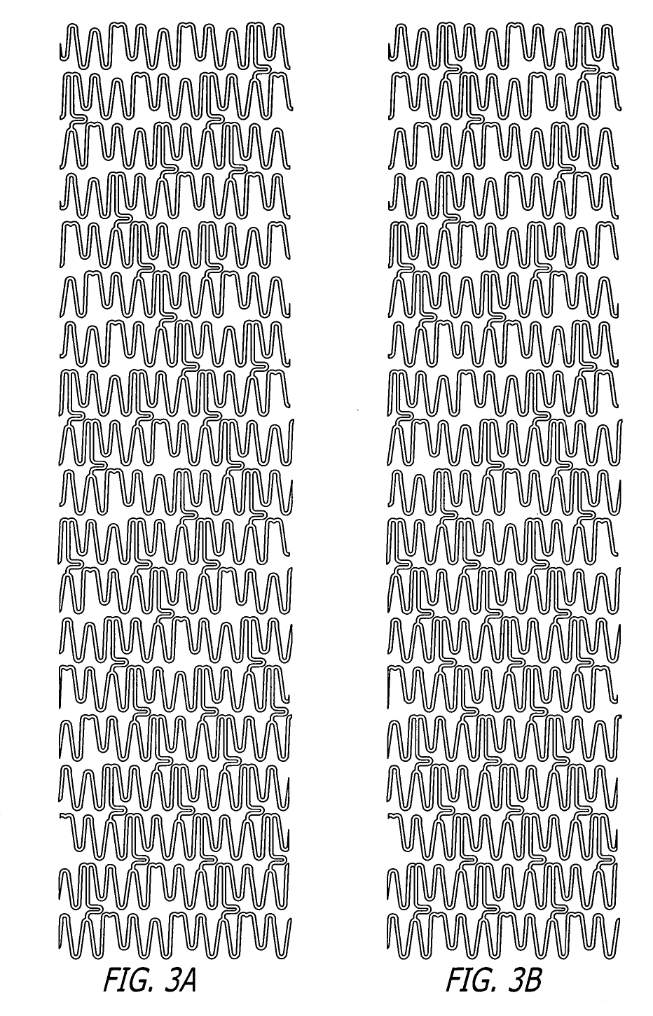

FIGS. 3A and 3B represent non-limiting closed (proximal; bottom of FIG. 3A and 3B) to open (distal; top of FIGS. 3A and 3B) embodiments of the present invention. Again, the stent configurations depicted in FIGS. 3A and 3B are different from the stent configurations depicted in FIGS. 1A-1C and the stent configurations depicted in FIGS. 2A and 2B. FIGS. 3A-3B depict the following non-limiting numbers of connectors at position along the length of the stent that create closed (proximal) to open (distal) embodiments of the present invention:

| Connector | No. of | No. of |
|---|---|---|
| Position | Connectors | Connectors |
| 1 | 1 | 2 |
| 2 | 2 | 1 |
| 3 | 2 | 1 |
| 4 | 1 | 1 |
| 5 | 2 | 2 |
| 6 | 1 | 2 |
| 7 | 2 | 1 |
| 8 | 3 | 2 |
| 9 | 2 | 2 |
| 10 | 2 | 2 |
| 11 | 3 | 3 |
| 12 | 1 | 3 |
| 13 | 2 | 3 |
| 14 | 3 | 2 |
| 15 | 2 | 3 |
| 16 | 3 | 3 |
| 17 | 3 | 3 |
| 18 | 2 | 3 |

As a guideline to determine whether a particular stent adopts a closed (proximal) to open (distal) embodiment of the present invention, the stent can be divided into two equal portions (i.e. the same number of crossover, connector or weld point positions). If the number of crossovers, connectors or weld points is counted in each portion, the closed proximal portion should have at least one more crossover, connector or weld point than the open distal portion. Alternatively, to determine if a particular stent adopts a closed (proximal) to open (distal) design, the stent can be divided into two portions and a percentage of actual crossovers, connectors or weld points over possible spaces for crossovers, connectors or weld points can be calculated. With this calculation method, the open end of the stent is required to have a smaller percentage of crossovers, connectors or weld points than the closed end.

These embodiments of the present invention have desirable characteristics when a particular treatment site requires a stent with enhanced deliverability characteristics. This embodiment might be chosen over an open-closed-open embodiment by a treating physician when the physician determines that the added support of a fully closed proximal end is desired at a particular treatment site.

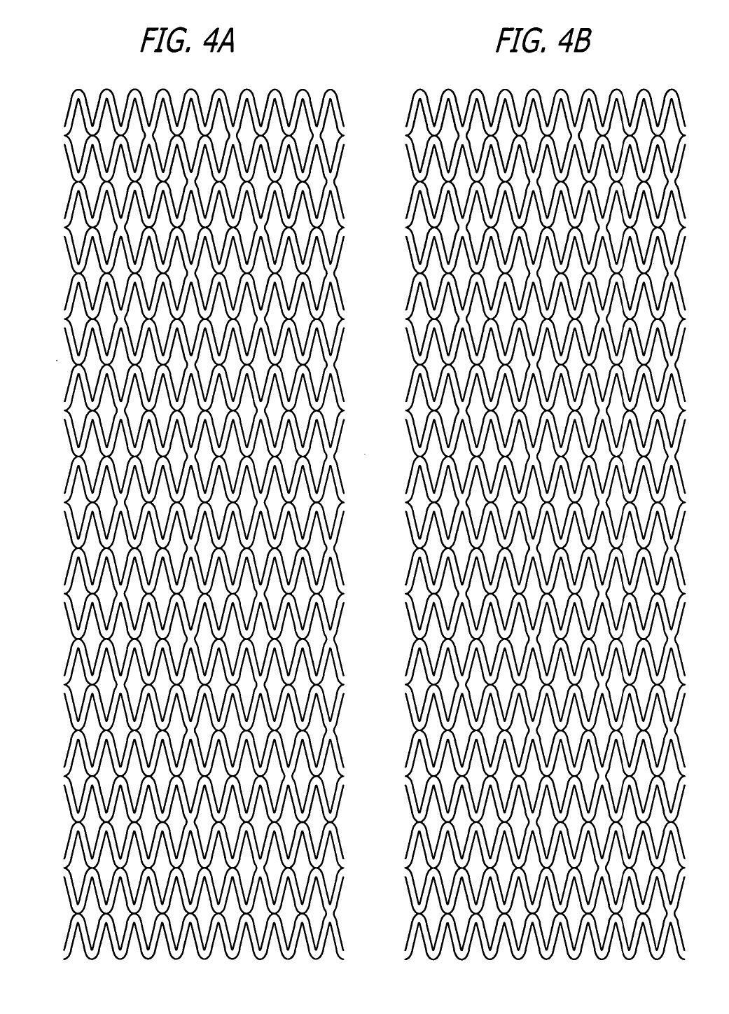

FIGS. 4A and 4B depict the following non-limiting numbers of crossovers at each crossover position along the length of the stent that create open (proximal; bottom of FIGS. 4A and 4B) to closed (distal; top of FIGS. 4A and 4B) embodiments of the present invention:

| Crossover | No. of | No. of |
|---|---|---|
| Position | Crossovers | Crossovers |
| 1 | 3 | 2 |
| 2 | 2 | 2 |
| 3 | 2 | 2 |
| 4 | 2 | 2 |
| 5 | 2 | 2 |
| 6 | 2 | 2 |
| 7 | 2 | 2 |
| 8 | 2 | 2 |
| 9 | 2 | 2 |
| 10 | 2 | 2 |
| 11 | 2 | 2 |
| 12 | 2 | 2 |
| 13 | 2 | 2 |
| 14 | 2 | 2 |
| 15 | 1 | 1 |
| 16 | 1 | 1 |
| 17 | 1 | 1 |
| 18 | 1 | 1 |

As a guideline to determine whether a particular stent adopts an open (proximal) to closed (distal) embodiment of the present invention, the stent can be divided into equal portions (i.e. the same number of crossover, connector or weld point positions). If the number of crossovers, connectors or weld points is counted in each portion, the open proximal portion should have at least one less crossover, connector or weld point than the closed distal portion. Alternatively, to determine if a particular stent adopts an open (proximal) to closed (distal) design, the stent can be divided into two portions and a percentage of actual crossovers, connectors or weld points over possible spaces for crossovers, connectors or weld points can be calculated. With this calculation method, the open end of the stent is required to have a smaller percentage of crossovers, connectors or weld points than the closed end.

These embodiments of the present invention have desirable characteristics when a particular treatment site is easily accessible. The closed portion of the stent provides required support while the open proximal end provides for a less abrupt transition from the stented to unstented proximal portion of the vessel. A less abrupt transition is especially advantageous at the proximal portion of the stented vessel because it is this area of the vessel that is otherwise most likely to suffer from restenosis.

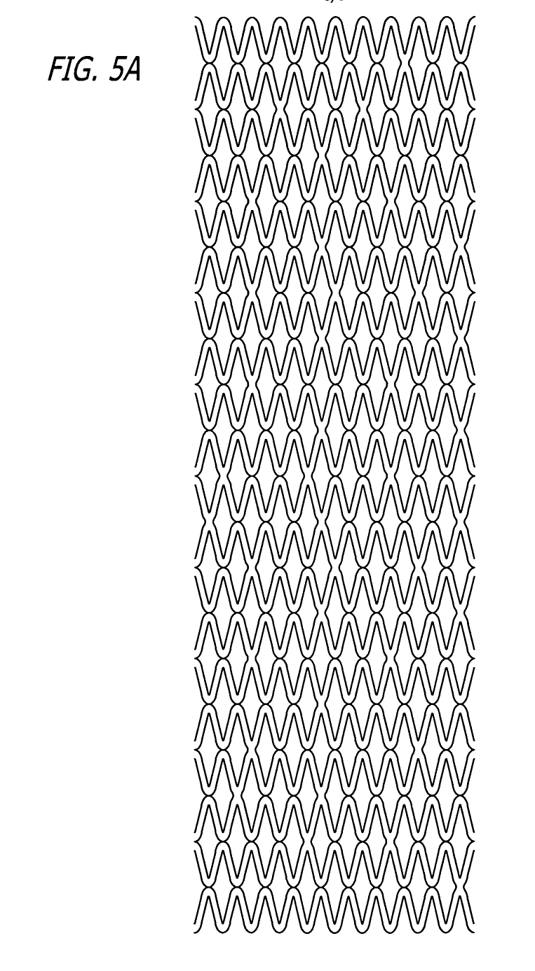

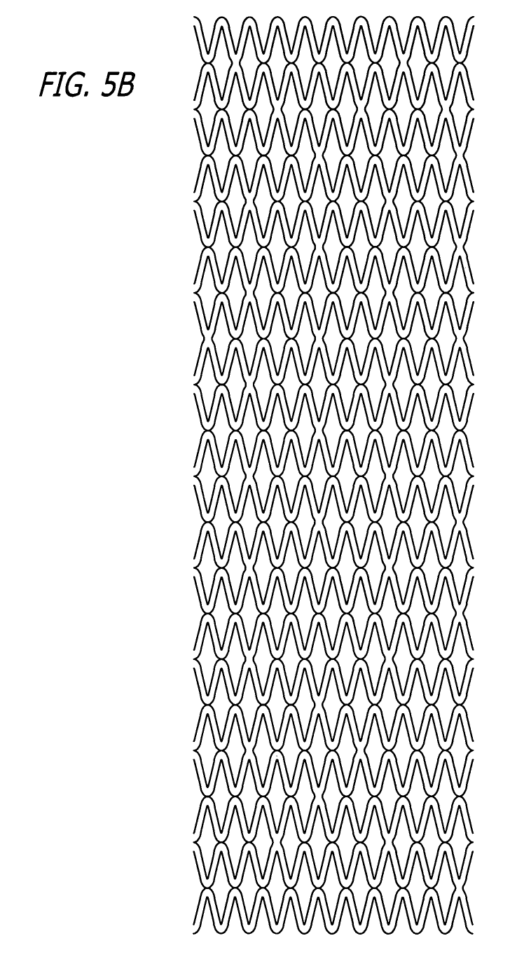

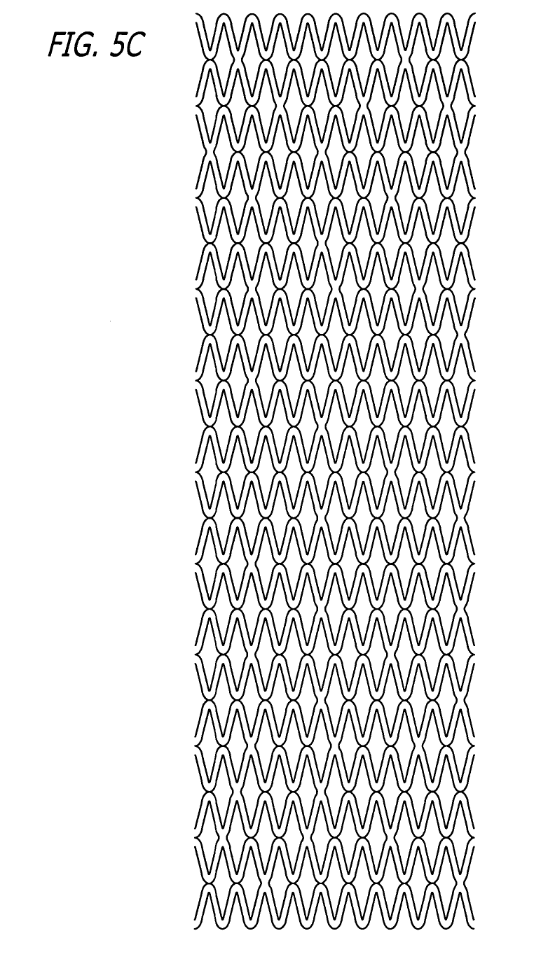

FIGS. 5A-5C depict the following non-limiting numbers of connectors at each crossover position along the length of the stent that create an open-closed-open embodiment (FIG. 5A); a open (proximal; bottom of 5B) to closed (distal; top of 5B) (FIG. 5B) and a closed-open-closed embodiment (FIG. 5C) of the present invention.

| Crossover | No. of | No. of | No. of |
|---|---|---|---|
| Position | Crossovers | Crossovers | Crossovers |
| 1 | 1 | 2 | 2 |
| 2 | 2 | 3 | 3 |
| 3 | 1 | 2 | 3 |
| 4 | 2 | 2 | 2 |
| 5 | 2 | 2 | 1 |
| 6 | 3 | 2 | 3 |
| 7 | 2 | 3 | 1 |
| 8 | 2 | 2 | 1 |
| 9 | 2 | 1 | 1 |
| 10 | 3 | 2 | 1 |
| 11 | 3 | 2 | 2 |
| 12 | 3 | 2 | 1 |
| 13 | 2 | 1 | 2 |
| 14 | 2 | 2 | 2 |
| 15 | 1 | 1 | 2 |
| 16 | 2 | 2 | 3 |
| 17 | 2 | 1 | 2 |
| 18 | 2 | 2 | 3 |
| 19 | 1 | 1 | 2 |

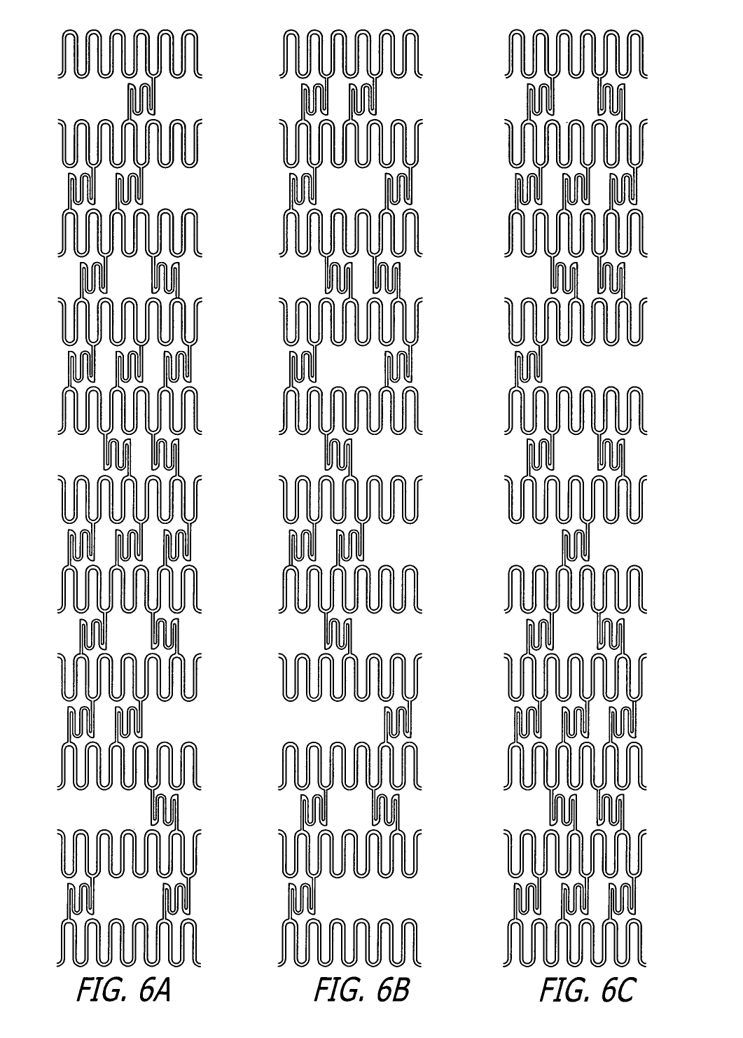

FIGS. 6A-6C depict additional embodiment of the stents of the present invention including another open-closed-open embodiment (FIG. 6A); another open (proximal; bottom of FIG. 6B) to closed (distal; top of 6B) embodiment (FIG. 6B) and another closed-open-closed embodiment (FIG. 6C). Specifically, FIGS. 6A-6C depict the following non-limiting numbers of connectors at each connector position along the length of the stent that create an open-closed-open embodiment (FIG. 6A); an open (proximal) to closed (distal) embodiment (FIG. 6B) and a closed-open-closed embodiment (FIG. 6C):

| Connector | No. of | No. of | No. of |
|---|---|---|---|
| Position | Connectors | Connectors | Connectors |
| 1 | 1 | 2 | 2 |
| 2 | 2 | 2 | 3 |
| 3 | 2 | 2 | 2 |
| 4 | 3 | 2 | 1 |
| 5 | 2 | 1 | 2 |
| 6 | 3 | 2 | 1 |
| 7 | 2 | 1 | 2 |
| 8 | 2 | 1 | 3 |
| 9 | 1 | 2 | 2 |
| 10 | 2 | 1 | 3 |

FIGS. 5A-5C and 6A-6C are provided to further illustrate that any stent configuration can be manufactured to create the various embodiments of the present invention.

The stents of the present invention can be used in any blood vessel, including, for example and without limitation, the coronary vasculature (which includes, without limitation, the right, left common, left anterior descending and circumflex arteries and their branches) and the peripheral vasculature (including, without limitation, branches of the carotid, aorta, femoral, renal, popliteal, and related arteries). While the stents of the present invention mainly have been described in terms of their use in a blood vessel, they can also be used in other lumens of the body, for example and without limitation, respiratory ducts, gastrointestinal ducts, bile ducts, the urinary system, the digestive tube, and the tubes of the reproductive system in both men and women.

The stents of the present invention can be coated with an appropriate material to enhance clinical performance. For instance, various coatings can be capable of releasing a drug or bioactive agent to assist in the repair of a diseased vessel and to assist in the prevention, treatment or inhibition of restenosis. Further, the stents of the present invention can be coated with a radiopaque material, such as a dye or marker to allow for better positioning during implantation. These coatings can be continuous or discontinious on the surface of the stents and can be disposed on the interior and/or the exterior surface(s) of the stents. Coatings can include one or more layers and can be coated either directly onto the stents or onto a primer material on the stents.

Any coating placed on the stents of the present invention should be biocompatible in order to minimize adverse interaction with the walls of the vessel or duct lumen or with the liquid flowing through the lumen. The coating can consist of a polymeric coating material. In one embodiment of the present invention the polymeric coating can have zwitterionic pendant groups, generally ammonium phosphate ester groups, for instance phosphoryl choline groups, or analogues thereof. Other examples of suitable polymers can be found in published International Patent Application Publication Nos. WO-A-93/16479 and WO-A-93/15775 which are hereby incorporated by reference. Coatings used in accordance with the present invention also can consist of nonpolymeric coating materials. The coating also can include a metallic coating placed onto the surface of the stent through electro- or electroless deposition processes.

Many substances that can enhance clinical performance can be included in coatings of the stents of the present invention. For instance, a radiopaque material, such as a dye or marker can be used to allow for better positioning during implantation. These markers can be placed on the ends of the stents as well as to mark the location of an open or closed portion of the stent. Drugs and bioactive agents that can enhance the clinical performance of the stents of the present invention also can be included. Examples of such drugs and bioactive agents include, for example and without limitation, antineoplastic, antinflammatory, antiplatelet, anticoagulant, antifibrin, antithromobin, antimitotic, antibiotic, antiproliferative and antioxidant substances, as well as calcium channel blockers, colchicine fibroblast growth factor antagonists, histamine antagonists, 3-hydroxy-3-methylglutaryl coenzyme A (HMG-CoA) reductase inhibitors, monoclonal antibodies, phosphodiesterase inhibitors, prostaglandin inhibitors, platelet-derived growth factor antagonists, serotonin inhibitors, steroids, and thioprotease inhibitors. Additional substances can include, for example and without limitation, rapamycin, cladribine, heparin, nitrous oxide, nitric oxide, actinomycin D, as well as, alpha-interferon, genetically engineered epithelial cells, and fish oil (omega 3-fatty acid).

It is to be understood that the present invention is not limited to the particular embodiments, materials, and examples described herein, as these can vary. It also is to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a stent” is a reference to one or more stents and includes equivalents thereof known to those skilled in the art and so forth.

Unless defined otherwise, all technical terms used herein have the same meanings as commonly understood by one of ordinary skill in the art to which this invention belongs. Specific methods, devices, and materials are described, although any methods and materials similar or equivalent to those described herein can be used in the practice or testing of the present invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A stent comprising adjacent sections, said sections being connected by crossovers, connectors or weld points wherein the number of said crossovers, connectors or weld points connecting said sections is varied between adjacent sections.

2. The stent according to claim 1, wherein said number of said crossovers, connectors or weld points is varied to create an open-closed-open stent.

3. The stent according to claim 2, wherein said open-closed-open stent has a first open end, a closed middle portion and a second open end wherein said first open end, said closed middle portion and said second open end all include the same number of positions for crossovers, connectors or weld points and wherein said first open end has fewer crossovers, connectors or weld points than said closed middle portion and said second open end has fewer crossovers, connectors or weld points than said closed middle portion.

4. The stent according to claim 2, wherein said open-closed-open stent has three portions: a first open end portion, a closed middle portion and a second open end portion wherein said open end portions each individually have a smaller percentage of crossovers, connectors or weld points than said closed middle portion wherein said percentage is the number of crossovers, connectors or weld points within a portion over the possible number of crossovers, connectors or weld points within said portion.

5. The stent according to claim 1, wherein said number of said crossovers, connectors or weld points is varied to create a closed-open-closed stent.

6. The stent according to claim 5, wherein said closed-open-closed stent has a first closed end portion, an open middle portion and a second closed end portion wherein said first closed end portion, said open middle portion and said second closed end portion all include the same number of positions for crossovers, connectors or weld points and wherein said first closed end portion has more crossovers, connectors or weld points than said open middle portion and said second closed end portion has more crossovers, connectors or weld points than said open middle portion.

7. The stent according to claim 5, wherein said closed-open-closed stent has three portions: a first closed end portion, an open middle portion and a second closed end portion wherein said closed end portions each individually have a greater percentage of crossovers, connectors or weld points than said open middle portion wherein said percentage is the number of crossovers, connectors or weld points within a portion over the possible number of crossovers, connectors or weld points within said portion.

8. The stent according to claim 1, wherein said number of said crossovers, connectors or weld points is varied to create an open (proximal) to closed (distal) stent.

9. The stent according to claim 8, wherein said open (proximal) to closed (distal) stent has an open portion and a closed portion wherein said open portion and said closed portion include the same number of positions for crossovers, connectors or weld points and wherein said open portion has fewer crossovers, connectors or weld points than said closed portion.

10. The stent according to claim 8, wherein said open (proximal) to closed (distal) stent has an open portion and a closed portion wherein said open portion has a smaller percentage of crossovers, connectors or weld points than said closed portion wherein said percentage is the number of crossovers, connectors or weld points within a portion over the possible number of crossovers, connectors or weld points within said portion.

11. The stent according to claim 1, wherein said number of said crossovers, connectors or weld points is varied to create a closed (proximal) to open (distal) stent.

12. The stent according to claim 11, wherein said closed (proximal) to open (distal) stent has an open portion and a closed portion wherein said open portion and said closed portion include the same number of positions for crossovers, connectors or weld points and wherein said open portion has fewer crossovers, connectors or weld points than said closed portion.

13. The stent according to claim 11, wherein said closed (proximal) to open (distal) stent has an open portion and a closed portion wherein said open portion has a smaller percentage of crossovers, connectors or weld points than said closed portion wherein said percentage is the number of crossovers, connectors or weld points within a portion over the possible number of crossovers, connectors or weld points within said portion.

14. A method of making a stent with varying characteristics along the length of the stent comprising varying the number of crossovers, connectors or weld points between adjacent sections of said stent.

15. The method according to claim 14, wherein the number of crossovers, connectors or weld points is varied to create an open-closed-open stent design.

16. The method according to claim 14, wherein the number of crossovers, connectors or weld points is varied to create a closed-open-closed stent design.

17. The method according to claim 14, wherein the number of crossovers, connectors or weld points is varied to create an open (proximal) to closed (distal) stent design.

18. The method according to claim 14, wherein the number of crossovers, connectors or weld points is varied to create a closed (proximal) to open (distal) stent design.
